(19) United States
(12) Patent Application Publication
(10) Pub. No.: US 2026/0201364 A1
(43) Pub. Date: Jul. 16, 2026

(54) Methods and Compositions for Obtaining Linked Functional and Sequence Data for Single Cells

(71) Applicant: Becton, Dickinson and Company, Franklin Lakes, NJ (US)

(72) Inventors: Narasimhan Jayanth VENKATACHARI, San Diego, CA (US); Xiaoshan Shirley SHI, Fremont, CA (US); Wei FAN, San Diego, CA (US); Yuming TANG, San Jose, CA (US); Edward Michael GOLDBERG, Redwood Estates, CA (US)

(21) Appl. No.: 19/136,030

(22) PCT Filed: Jan. 26, 2024
(86) PCT No.: PCT/US2024/013134

§ 371 Date: Jun. 5, 2025

Related U.S. Application Data
(60) Provisional application No. 63/442,227, filed on Jan. 31, 2023.

(51) Int. Cl. C12N 15/10

(52) U.S. Cl. CPC C12N 15/1065

(57) ABSTRACT

Methods of obtaining linked functional and sequence data for single cells, e.g., of a cellular sample, are provided. Aspects of the methods include functionally assaying partitioned single cells; visually indexing functionally-assayed, partitioned single cells using unique combinations of distinct nucleic acid-barcoded identifying particles; obtaining sequence data for the visually-indexed, functionally-assayed, partitioned single cells; and linking functional and sequence data for sequenced, visually-indexed, functionally-assayed, partitioned single cells. Also provided are compositions for practicing methods of the invention.

### CROSS REFERENCE TO RELATED APPLICATION

This application claims priority to the filing date of U.S. Provisional Patent Application Ser. No. 63/442,227 filed on Jan. 31, 2023; the disclosure of which application is incorporated herein by reference.

### INTRODUCTION

Current technology allows for measurement of gene expression of single cells in a massively parallel manner (e.g., >10,000 cells) by attaching cell specific oligonucleotide barcodes to poly(A) mRNA molecules from individual cells as each of the cells is co-localized with a barcoded reagent bead in a compartment. One platform that allows measurement of gene expression of single cells in a massively parallel manner is the BD Rhapsody™ Single-Cell Analysis System. The BD Rhapsody™ Single-Cell Analysis System is a platform that allows high-throughput capture of nucleic acids from single cells using a simple cartridge workflow and a multitier barcoding system. The resulting captured information can be used to generate various types of next-generation sequencing (NGS) libraries, including libraries suitable for whole transcriptome analysis, e.g., for discovery biology and targeted RNA analysis for high sensitivity transcript detection. Shum et al., “Quantitation of mRNA Transcripts and Proteins Using the BD Rhapsody™ Single-Cell Analysis System,” Adv Exp Med Biol. 2019; 1129:63-79.

Gene expression may affect protein expression. Protein-protein interaction may affect gene expression and protein expression. As such, more recently systems and methods that can quantitatively analyze protein expression in cells, and simultaneously measure protein expression and gene expression in cells, have been developed. One such platform is the BD Abseq platform. AbSeq is a method to profile proteins in single cells. In Abseq, the usual fluorophore labeled antibodies are replaced with nucleic acid sequence tags that can be read out at the single-cell level, e.g., via barcoding and NGS sequencing. “The objective of Abseq is to enable the sensitive, accurate, and comprehensive characterization of proteins in large numbers of single cells. Cells are bound with antibodies against the different target epitopes, as in conventional immunostaining, except that the antibodies are labeled with unique sequence tags. When an antibody binds its target, the DNA tag is carried with it, allowing the presence of the target to be inferred based on the presence of the tag. In this way, counting tags provides an estimate of the different epitopes present in the cell, as detected via antibody binding.” Shahi et al., “Abseq: Ultrahigh-throughput single cell protein profiling with droplet microfluidic barcoding. Sci Rep 7, 44447 (2017).”

### SUMMARY

The inventors have realized that, while combining protein expression data along with transcriptome data, e.g., as is done with AbSeq, has provided significant insight into single cells, multiple genes, post transcriptional and post translational factors, as well as signaling pathways regulate, cell function. Understanding the omics data in relation to cellular function and phenotype would be valuable to the further understanding of single cells, as well as provide for the development of better strategies in translational research, including identifying novel biomarkers, developing diagnostic assays and exploring novel therapeutics and clinical solutions. As such, the inventors have realized the need for providing ways to link functional and sequence data, e.g., expression and/or transcriptome data, in single cells. Embodiments of the invention satisfy this need.

Methods of obtaining linked functional and sequence data for single cells, e.g., of a cellular sample, are provided. Aspects of the methods include functionally assaying partitioned single cells; visually indexing functionally-assayed, partitioned single cells using unique combinations of distinct nucleic acid-barcoded identifying particles; obtaining sequence data for the visually-indexed, functionally-assayed, partitioned single cells; and linking functional and sequence data for sequenced, visually-indexed, functionally-assayed, partitioned single cells. Also provided are compositions for practicing methods of the invention.

### BRIEF DESCRIPTION OF THE FIGURES

The invention may best be understood from the following detailed description when read in conjunction with the accompanying drawings. Included in the drawings are the following figures:

FIGS. 1A and 1B provide schematic representations of cell-binding beads according to embodiments of the invention.

FIG. 2 provides a schematic representation of three distinct nucleic acid-barcoded identifying particles (Pheno Seq particles) in accordance with an embodiment of the invention.

FIGS. 3A and 3B provide a view of partitions visually indexed with unique combinations of identifying particles, in accordance with two different embodiments of the invention.

FIG. 4 illustrates how cell-binding bead nucleic acids hybridized to both cell capture bead nucleic acid and identifying particle nucleic acids, in accordance with an embodiments of the invention.

FIGS. 5A and 5B illustrate aspects of library preparation, in accordance with an embodiment of the invention.

FIG. 6 illustrates how functional data is matched with sequence data, in accordance with an embodiment of the invention.

FIG. 7 illustrates a workflow in accordance with an embodiment of the invention.

### DEFINITIONS

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, an antibody can be a full-length (e.g., naturally occurring or formed by normal immunoglobulin gene fragment recombinatorial processes) immunoglobulin molecule (e.g., an lgG antibody) or an immunologically active (i.e., specifically binding) portion of an immunoglobulin molecule, like an antibody fragment. In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein nis 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido (5,4-b) (1,4)benzoxazin-2 (3H)-one), phenothiazine cytidine (1H-pyrimido (5,4-b) (1,4)benzothiazin-2 (3H)-one), G-clamps such as a substituted phenoxazine cytidine (e.g., 9-(2-aminoethoxy)-H-pyrimido (5,4-(b)(1,4)benzoxazin-2 (3H)-one), phenothiazine cytidine (1H-pyrimido (5,4-b) (1,4)benzothiazin-2 (3H)-one), G-clamps such as a substituted phenoxazine cytidine (e.g., 9-(2-aminoethoxy)-H-pyrimido (5,4-(b)(1,4)benzoxazin-2 (3H)-one), carbazole cytidine (2H-pyrimido (4,5-b) indol-2-one), pyridoindole cytidine (H-pyrido (3′,2′: 4,5) pyrrolo[2,3-d]pyrimidin-2-one).

As used herein, the term “sample” can refer to a composition comprising targets. Suitable samples for analysis by the disclosed methods, devices, and systems include cells, tissues, organs, or organisms. A cellular sample is a composition that is made up of multiple cells, such as a composition that includes multiple disparate cells, such as an aqueous composition of single cells, where the number of cells may vary.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which nucleic acids may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the terms “bead” and “particle”.

As used here, the term “target” can refer to a composition which can be analyzed in accordance with embodiments of the invention. Exemplary suitable targets for analysis by the disclosed methods, devices, and systems include oligonucleotides, DNA, RNA, mRNA, microRNA, tRNA, and the like. Targets can be single or double stranded. In some embodiments, targets can be proteins, peptides, or polypeptides. In some embodiments, targets are lipids. As used herein, “target” can be used interchangeably with “species.”

As used herein, the term “reverse transcriptases” can refer to a group of enzymes having reverse transcriptase activity (i.e., that catalyze synthesis of DNA from a RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transciptases, and group II intron reverse transcriptases. Examples of group II intron reverse transcriptases include the Lactococcus lactis LI.LtrB intron reverse transcriptase, the Thermosynechococcus elongatus Tel4c intron reverse transcriptase, or the Geobacillus stearothermophilus Gsl-IIC intron reverse transcriptase. Other classes of reverse transcriptases can include many classes of non-retroviral reverse transcriptases (i.e., retrons, group II introns, and diversity-generating retroelements among others).

### DETAILED DESCRIPTION

Methods of obtaining linked functional and sequence data for single cells, e.g., of a cellular sample, are provided. Aspects of the methods include functionally assaying partitioned single cells; visually indexing functionally-assayed, partitioned single cells using unique combinations of distinct nucleic acid-barcoded identifying particles; obtaining sequence data for the visually-indexed, functionally-assayed, partitioned single cells; and linking functional and sequence data for sequenced, visually-indexed, functionally-assayed, partitioned single cells. Also provided are compositions for practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of obtaining linked functional and sequence data for single cells, e.g., of an initial cellular sample, are provided. By “linked functional and sequence data” is meant a combined data set that includes both functional data and nucleic acid sequence data that can be attributed to the same cell, such that the two types of data can be considered as originating from the same cell. In other words, linked functional and sequence data is a data set the includes both functional data and nucleic acid sequence data that is obtained from the same cell. Functional data is data obtained from a cell using a functional assay (i.e., a phenotypic assay), where examples of functional data that may be obtained for single cells and linked to sequence data in embodiments of the invention are further described in greater detail below. Nucleic acid sequence data refers to data obtained using a nucleic acid sequencing technique, which identifies the sequence of nucleotides in nucleic acid molecules. Nucleic acid sequencing data from a cell includes the sequence of one or more nucleic acid sequences, e.g., RNA molecules, present in the cell. Such data may include gene expression data. Such data may also include protein expression data data, e.g., as may be obtained using AbSeq. In some instances, the sequence data may be multi-omic data. Such data may be obtained using a variety of sequence protocols, including next generation sequence (NGS) protocols.

As summarized above, aspects of the methods include: (a) functionally assaying partitioned single cells; (b) visually indexing functionally-assayed, partitioned single cells using unique combinations of distinct nucleic acid-barcoded identifying particles; (c) obtaining sequence data for the visually-indexed, functionally-assayed, partitioned single cells; and (d) linking functional and sequence data for sequenced, visually-indexed, functionally-assayed, partitioned single cells. Embodiments of each of these steps are now described in greater detail.

### Functionally Assaying Partitioned Single Cells

In embodiments of the invention, aspects of the methods include functionally assaying partitioned single cells. By “functionally assaying partitioned single cells” is meant that single cells that are partitioned from each other are functionally assayed, e.g., assayed for one or more phenotypic characteristics. In embodiments, single cells are provided in compartments that are fluidically isolated from other compartments, and functionally assayed in the compartments. Embodiments of functionally assaying partitioned single cells include: (a) contacting cells of a cellular sample with nucleic acid-barcoded, cell-binding beads to produce bead-bound cells; (b) partitioning the bead-bound cells to produce partitioned bead-bound single cells; and (c) functionally assaying the partitioned, bead-bound, single cells to obtain functional data for the partitioned, bead-bound, single cells.

### Cellular Sample

Single cells that are functionally assayed in embodiments of the invention may be cells that are initially present in a cellular sample. While the number of cells in a given cellular sample may vary, in some instances the number of cells ranges from 50 to 50,000,000, such as 100 to 1,000,000 and including 500 to 100,000. Cells present in a given cellular sample may be any type of cell, including prokaryotic and eukaryotic cells. Suitable prokaryotic cells include, but are not limited to, bacteria such as E. coli, various Bacillus species, and the extremophile bacteria such as thermophiles, etc. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tulapia, tuna, carp, flounder, halibut, swordfish, cod and zebrafish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes. Suitable cells also include those cell types implicated in a wide variety of disease conditions, even while in a non-diseased state. Accordingly, suitable eukaryotic cell types include, but are not limited to, tumor cells of all types (e.g., melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. In certain embodiments, the cells are primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In certain embodiments, the cells used in the present invention are taken from a subject. As used herein “subject” refers to both human and other animals as well as other organisms, such as experimental animals. Thus, the methods and compositions described herein are applicable to both human and veterinary applications. In certain embodiments the subject is a mammal, including embodiments in which the subject is a human patient either having (or suspected of having) a disease or pathological condition.

In certain embodiments, the cells being analyzed are enriched prior to indexing, e.g., as described in greater detail below. For example, if the cells of interest are white blood cells derived from a human subject, whole blood from the subject may be subjected to density gradient centrifugation to enrich for peripheral blood mononuclear cells (PBMCs, or white blood cells). Cells may be enriched using any convenient method known in the art, including fluorescence activated cell sorting (FACS), magnetically activated cell sorting (MACS), density gradient centrifugation and the like. Parameters employed for enriching certain cells from a mixed population include, but are not limited to, physical parameters (e.g., size, shape, density, etc.), in vitro growth characteristics (e.g., in response to specific nutrients in cell culture), and molecule expression (e.g., expression of cell surface proteins or carbohydrates, reporter molecules, e.g., green fluorescent protein, etc.).

In certain embodiments, the cells are live cells which retain viability during the course of the assay. By “retain viability” is meant that a certain percentage of the cells remain alive at the conclusion of the assay, including from about 20% viable up to and including about 100% viable. In certain other embodiments, the methods of the present invention are carried out in such a manner as the cells are rendered non-viable during the course of the assay, e.g., the cells may be fixed, permeabilized, or otherwise maintained in buffers or under conditions in which the cells do not survive. Such parameters are generally dictated by the nature of the assay being performed as well as the reagents being employed.

In some instances, the cells may be treated, e.g., with a stimulus. Stimuli with which cells may be treated may vary, ranging from culture conditions, exposure to changes in temperature, e.g., heat or cold, exposure to electromagnetic radiation, e.g., light, exposure to active agents, exposure to mechanical changes, etc. As desired, different cellular samples of the plurality may be treated with the same or different stimulus. As such, in some instances the method includes differentially treating two or more of the plurality of cellular samples, e.g., where two or more different sample are contacted with different active agents, or different concentrations of the same active agent, etc.

Contacting Cells with Nucleic Acid-Barcoded, Cell-Binding Beads

In embodiments, the methods include contacting cells of a cellular sample with nucleic acid-barcoded, cell-binding beads to produce bead-bound cells. The nucleic acid-barcoded, cell-binding beads that are contacted with cells of the cellular sample may vary, and in some instances include: a bead; a specific binding member; and a cell-binding bead nucleic acid comprising a barcode.

The bead component of the nucleic acid-barcoded, cell binding beads may vary as desired, and may be any solid support, e.g., a discrete solid or semi-solid surface, to which nucleic acids may be attached. The bead may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). The bead may comprise a discrete particle that may be spherical (e.g., microspheres) or have a non-spherical or irregular shape, such as cubic, cuboid, pyramidal, cylindrical, conical, oblong, or disc-shaped, and the like. The bead can be non-spherical in shape. In some instances, the bead may be magnetic. Further details regarding beads that may be employed in embodiments of the invention may be found in in U.S. Patent Application Publication No. US2018/0088112; US Patent Application Publication No. 2018/0200710; U.S. Patent Application Publication No. US2018/0346970; U.S Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

Beads may display a specific binding member on a surface thereof. The specific binding member components of the nucleic acid-barcoded, cell-binding beads employed in embodiments of the invention may vary. The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. Specific binding members may vary, where examples of specific binding members include, but are not limited to, polypeptides, nucleic acids, carbohydrates, lipids, peptoids, etc. In some instances, the specific binding member is proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specifically binds to a epitope, e.g., of a cell surface protein. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions. In certain embodiments, the specific binding member is a Fab fragment, a F(ab′)2 fragment, a scFv, a diabody or a friabody. In certain embodiments, the specific binding member is an antibody. In some cases, the specific binding member is a murine antibody or binding fragment thereof. In certain instances, the specific binding member is a recombinant antibody or binding fragment thereof.

The specific binding member component of the nucleic acid-barcoded, cell-binding beads may specifically bind to any convenient cell marker. In some instances, the specific binding member binds to cell surface markers, where cell surface markers of interest include, but are not limited to, ubiquitous cell surface markers, i.e., cell surface markers that are at least predicted to be on all cells of a given cellular sample to be processed in a given workflow in accordance with the present invention. Examples of ubiquitous cell surface markers to which specific binding member/oligonucleotide sub-barcodes may specific bind include, but are not limited to: CD44, CD45, β-2 micro-globulin, and the like.

The nucleic acid-barcoded, cell-binding beads employed in embodiments of the invention also include a cell-binding bead nucleic acid that includes a barcode component. Barcode components may vary in length, ranging in some instances from 10 to 500 nt, such as 15 to 100 nt. In some instances, the barcode components may be made up of ribonucleic acids or deoxyribonucleic acids, as desired. Barcode components of embodiments of the invention may include a barcode region or domain, as well as other domains that find use in embodiments of the invention, where such domains may include a bead identifier domain, a capture sequence, a primer binding site, a domain complementary to domains in identifying particle nucleic acids, etc. The barcode component may be covalently bound directly to a bead, or via an intermediate group, e.g., a disulfide bond, as desired. In certain embodiments, the cell-binding bead nucleic acid may be linked to the bead by a cleavable linker, which linker may be cleaved by cell lysis conditions, where examples of such linkers include, but are not limited to: disulfide linkers, and the like.

A barcode region (i.e., barcode domain) of barcode component is a domain or subsequence, i.e., stretch, of the barcode components that serves as an identifier of the bead to which it is attached. The sequence of a given barcode region can be employed as an identifier of the bead with which the barcode is associated, such that amplicons having the barcode can be assigned as deriving from a nucleic acid-barcoded, cell-binding bead. As such, the sequence of barcode region corresponds to the bead to which it is attached. The barcode region may have any convenient sequence and may vary in length. The barcode can be, for example, a nucleotide sequence having any suitable length, for example, from about 4 nucleotides to about 200 nucleotides. In some embodiments, the barcode region is a nucleotide sequence of 25 nucleotides to about 45 nucleotides in length. In some embodiments, the barcode region can have a length that is, is about, is less than, is greater than, 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 70 nucleotides, 80 nucleotides, 90 nucleotides, 100 nucleotides, 200 nucleotides, or a range that is between any two of the above values.

The barcode component may include a first domain complementary to a target binding region of a nucleic acid capture bead, which domain may be referred to as a capture sequence. The capture sequence is a domain or region that serves as a binding site for target binding region, e.g., of a bead bound nucleic acid of a cell capture bead, such as described below. Capture sequences of interest may vary, as desired, and may be specific or random or semi random. In some instances, the capture sequence is a sequence that hybridizes to a target binding region of a bead bound nucleic acid of a cell capture bead, e.g., as described in greater detail below. In some instances, the capture sequence is a poly(A) sequence, which poly(A) sequence is configured to hybridize to an oligodT target binding region, such as described in greater detail below. In such instances, the length of the poly(A) sicapture sequence may vary, ranging in some instances from 3 to 50 nt, such as 5 to 25 nt. When present, the capture sequence may be positioned 3′ of barcode domain. In some instances, the capture sequence is positioned at the 3′ end of the cell-binding bead nucleic acid.

In some instances, the barcode component further includes a second domain complementary to a sequence present in identifying particle nucleic acids of the nucleic acid-barcoded identifying particles, e.g., as described in greater detail below. The sequence of this second domain is selected to hybridize to a sequence present in identifying particle nucleic acids, and may have any convenient sequence. The length of this second domain sequence may vary, ranging in some instances from 3 to 50, such as 5 to 25 nt. When present, the second domain may be positioned 3′ of barcode domain, as desired. In some instances, this second domain may be positioned 3′ of the capture sequence (i.e., first domain), where in some instances this second domain is present at the 3′ end of the barcode component. In other instances, this second domain is positioned 3′ of the barcode domain but 5′ of the capture sequence (first domain).

Barcode components may further include a primer binding site. A primer binding site, when present, may be configured to bind to a primer employed, e.g., in preparing sequenceable nucleic acids. For example, a barcode component may include a primer binding site that is common to all nucleic acid-barcoded, cell-binding beads employed in a given workflow. This primer binding site may be different from other primer binding sites employed, e.g., in preparing sequenceable libraries, such as universal primer binding sites, e.g., as described in greater below. In some instances, a primer binding site can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A primer binding site can vary in length, and can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. The primer binding site can be positioned at the 5′ end of the barcode component. In some instances, the primer binding site can be the same primer binding site that is present on an oligonucleotide labeled cellular component binding reagent, e.g., as described in greater detail below, such as a primer binding site found in AbSeq oligonucleotide labeled antibodies.

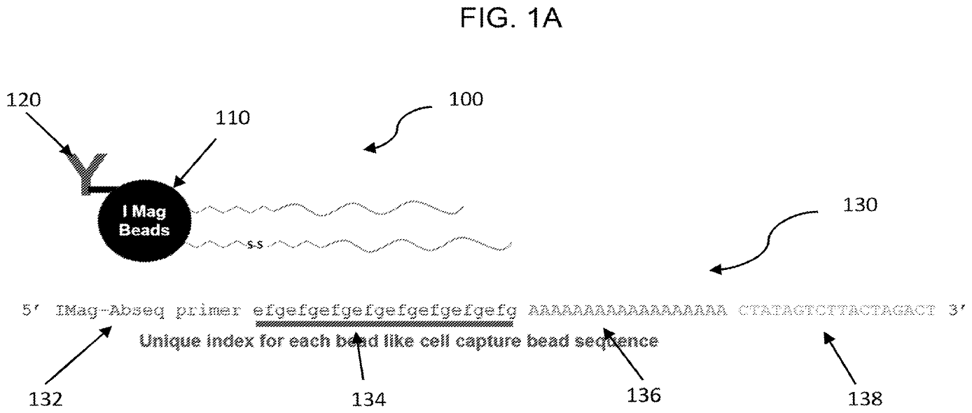

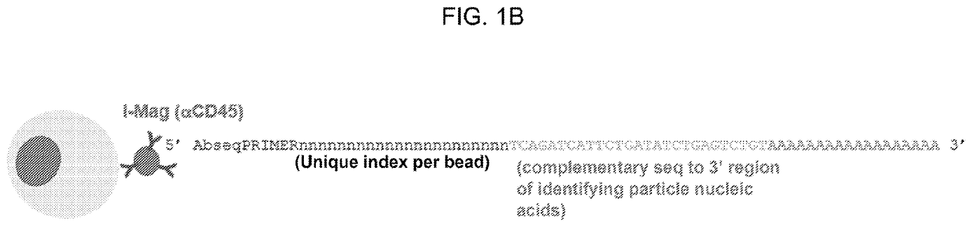

FIG. 1A provides a schematic of a nucleic acid-barcoded, cell-binding bead that may be employed in embodiments of the invention. As illustrated in FIG. 1, nucleic acid-barcoded, cell-binding bead 100 includes a magnetic bead 110 having conjugated thereto an antibody 120 specific for a cell surface marker, e.g., CD45. Also present is barcode component 130 that includes, going from the 5′ to the 3′ end, a primer binding site 132, a barcode region 134, a first domain (i.e., capture sequence) 136 and a second domain 138 complementary to a sequence present in identifying particle nucleic acids of the nucleic acid-barcoded identifying particles, e.g., as described in greater detail below. As illustrated, the barcode component 130 may be covalently bound directly to a bead, or via an intermediate group, e.g., a disulfide bond, as desired. FIG. 1B provides a schematic of an alternative embodiment of a nucleic acid-barcoded, cell-binding bead that may be employed in embodiments of the invention. As illustrated in FIG. 1B, the barcode component has the capture sequence positioned at the 3′ end, with second domain positioned between the capture sequence and the barcode domain.

Cells of a cellular sample may be contacted with nucleic acid-barcoded, cell-binding beads to produce bead-bound cells using any convenient protocol. The cells may be contacted with the beads under conditions sufficient for the specific binding members, e.g., antibodies, displayed on the surface of the beads to specifically bind to corresponding epitopes present on cells of the cellular sample, so as to produce a composition of bead bound cells. The number of beads bound to a cell in a given bead-bound cell may vary, ranging in some instances from 1 to 5, such as 1 to 2. Among the bead-bound cells, the barcode components may share common domains, such as the primer binding site, first and/or second domains as desired.

### Partitioning the Bead-Bound Cells

Following production of bead-bound cells, e.g., as described above, embodiments of the methods include partitioning the bead-bound cells to produce partitioned bead-bound single cells, where each of the partitioned bead-bound cells is stably associated with one on more nucleic acid-barcoded cell-binding beads, e.g., via specific binding of a specific binding member, e.g., antibody, of the bead, to an epitope, e.g., of a cell surface protein, of the cell. As such, following production of the bead-bound cells, e.g., as described, embodiments of the methods include partitioning the bead-bound cells to produce partitioned, bead-bound, single cells. In some instances, the partitioning includes distributing the bead-bound cells into partitions or compartments so that compartments include single bead-bound cells, i.e., compartments only include one bead-bound cell. By “partitioning” is meant that the bead-bound cells are placed into small reaction chambers, which may be fluidically isolated structures defined by solid materials, such as microwells, configured to accommodate the bead-bound cells. In some embodiments of the disclosed methods, devices, and systems, a plurality of microwells that are randomly distributed across a substrate are used. In some embodiments, the plurality of microwells is distributed across a substrate in an ordered pattern, e.g., an ordered array. In some embodiments, a plurality of microwells is distributed across a substrate in a random pattern, e.g., a random array. The microwells may be configured in a variety of shapes and sizes. Appropriate well geometries include, but are not limited to, cylindrical, elliptical, cubic, conical, hemispherical, rectangular, or polyhedral, e.g., three dimensional geometries comprised of several planar faces, for example, rectangular cuboid, hexagonal columns, octagonal columns, inverted triangular pyramids, inverted square pyramids, inverted pentagonal pyramids, inverted hexagonal pyramids, or inverted truncated pyramids. In some embodiments, non-cylindrical microwells, e.g., wells having an elliptical or square footprint, may offer advantages in terms of being able to accommodate larger cells. In some embodiments, the upper and/or lower edges of the well walls may be rounded to avoid sharp corners and thereby decrease electrostatic forces that may arise at sharp edges or points due to concentration of electrostatic fields. Thus, use of rounded off corners may improve the ability to retrieve beads from the microwells. Microwell dimensions may be characterized in terms of absolute dimensions. In some instances, the average diameter of the microwells may range from about 5 μm to about 100 μm. In other embodiments, the average microwell diameter is at least 5 μm, at least 10 μm, at least 15 μm, at least 20 μm, at least 25 μm, at least 30 μm, at least 35 μm, at least 40 μm, at least 45 μm, at least 50 μm, at least 60 μm, at least 70 μm, at least 80 μm, at least 90 μm, or at least 100 μm. In yet other embodiments, the average microwell diameter is at most 100 μm, at most 90 μm, at most 80 μm, at most 70 μm, at most 60 μm, at most 50 μm, at most 45 μm, at most 40 μm, at most 35 μm, at most 30 μm, at most 25 μm, at most 20 μm, at most 15 μm, at most 10 μm, or at most 5 μm. The volumes of the microwells used in the methods of the invention may vary, ranging in some instances from about 200 μm³ to about 800,000 μm³. In some embodiments, the micro well volume is at least 200 μm³, at least 500 μm³, at least 1,000 μm³, at least 10,000 μm³, at least 25,000 μm³, at least 50,000 μm³, at least 100,000 μm³, at least 200,000 μm³, at least 300,000 μm³, at least 400,000 μm³, at least 500,000 μm³, at least 600,000 μm³, at least 700,000 μm³, or at least 800,000 μm³. In other embodiments, the microwell volume is at most 800,000 μm³, at most 700,000 μm³, at most 600,000 μm³, 500,000 μm³, at most 400,000 μm³, at most 300,000 μm³, at most 200,000 μm³, at most 100,000 μm³, at most 50,000 μm³, at most 25,000 μm³, at most 10,000 μm³, at most 1,000 μm³, at most 500 μm³, or at most 200 μm³. The number of microwells in a given device employed in embodiments of the invention may vary, where in some instances the number is 100 or more, such as 250 or more, e.g., 500 or more, including 1000 or more, such as 5,000 or more, e.g., 10,000 or more, wherein some instances the number is 15,000 or less, e.g., 12,500 or less. Microwells suitable for use in embodiments of the invention are further described in PCT application serial no. PCT/US2016/014612 published as WO/2016/118915, the disclosure of which is herein incorporated by reference. As used herein, a substrate can refer to a type of solid support. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. While the number of wells, e.g., microwells, in a well plate, e.g., microwell array, may vary in a given partitioning step, in some instances the number is 100 or more, such as 250 or more, e.g., 500 or more, including 1000 or more, such as 5,000 or more, e.g., 10,000 or more, 100,000 or more, including 250,000 or more, wherein some instances the number is 500,000 or less, such as 400,000 or less, and in some instances 15,000 or less, e.g., 12,500 or less.

In partitioning bead-bound cells, the bead-bound cells may be positioned in compartments, e.g., microwells of a microwell array, using any convenient protocol. The disclosure provides for methods for compartmentalizing the bead-bound cells into partitions in order to partition the bead-bound cells. A collection of bead-bound cells, for example, can be introduced into structures, e.g., microwells, to partition the bead-bound cells. The bead-bound cells can be contacted with compartments, for example, by gravity flow wherein bead-bound cells can settle into the partitioning structures. In some instances, an aqueous composition of the bead-bound cells is contacted with, e.g., by flowing it across, an array of microwells such that bead-bound cells are deposited into the microwells. The aqueous composition that includes the bead-bound cells may be flowed through a flow cell in fluidic communication with the microwells. Suitable protocols and systems for partitioning the bead-bound cells into microwells are described in PCT application serial no. PCT/US2016/014612 published as WO/2016/118915, the disclosure of which is herein incorporated by reference. To partition the bead-bound cells, any convenient protocol may be employed, e.g., dispensing, such as pipetting, aliquots of the bead-bound cells into the compartments, flowing sample over the surface of the well plate, and the like.

### Functionally Assaying Partitioned, Bead-Bound, Single Cells

Following production of partitioned, bead-bound, single cells, e.g., as described above, embodiments of the methods include functionally assaying the partitioned, bead-bound, single cells to obtain functional data for the partitioned, bead-bound, single cells. Functional assays that may be performed on the partitioned, bead-bound single cells may vary. In some instances, functionally assaying the partitioned, bead-bound single cells to obtain functional data for the partitioned, bead-bound single cells includes evaluating partitioned bead-bound single cells over time, which time may vary, ranging in some instances from 1 sec to 24 hour or Ionger, such as 1 sec to 12 hours, including 30 sec to 6 hours. In some instances, functionally assaying the partitioned, bead-bound single cells to obtain functional data for the partitioned, bead-bound single cells includes evaluating partitioned, bead-bound single cells in response to a stimulus. Stimuli employed in such instances may vary, where examples of stimuli include, but are not limited to, chemical stimulus, mechanical stimulus, physical stimulus or combinations thereof. The evaluation may detect changes arising from any number of sources, e.g., production of signal from a signal production system (e.g., reagent reporter system), changes in morphology, and the like.

Functional assays that may be performed on partitioned, bead-bound, single cells include, but are not limited to: target cell lysis assays; cell chemotaxis assays; single cell secretome assays, including real time single cell scretome assays, CAR-T cell evaluation and characterization assays, single cell phenotype characterization (cellular sub compartment) assays, such as lysosomal activation, endocytosis, phagocytosis, autophagy, calcium signaling, Akt, NFκB translocation, and reporter assays; assays that evaluate the effect of small molecules (such as novel therapeutics), e.g., in tumor microenvironments; single cell antibody production assays; dendritic cell maturation, macrophage differentiation, cellular differentiation-change in morphology assays; neuronal differentiation assays; and virus production and regulation assays.

Functional assays performed on partitioned, bead-bound, single cells generate functional data for those cells. The resultant functional data may be recorded as arising from a particular partition, and therefore cell located therein, and then linked to that partition and sequence data obtained for cell therein using the visual index obtained for that partition, as described in greater detail below.

### Visually Indexing Functionally-Assayed, Partitioned Single Cells

Following obtainment of functional data for the partitioned, bead-bound, single cells, e.g., as described above, embodiments of the methods include visually indexing functionally-assayed, partitioned, single cells. By “visually indexing” is mean obtaining indexing image data for each partition, which indexing image data can be used to correlate functional data obtained from a cell in a given partition with sequencing data obtained for the cell in that same partition, e.g., as described in greater detail below. Visual indexing may include obtaining image data (i.e., indexing image data) for partitions that include functionally assayed signals, and specifically image data of unique combinations of identifying particles present in partitions. In some instances, indexing image data is obtained using unique combinations of distinct nucleic acid-barcoded identifying particles. In such instances, methods include introducing unique combinations of distinct nucleic acid-barcoded identifying particles to partitions that include functionally-assayed, bead-bound single cells to produce indexed partitions, where the indexed partitions include: a functionally assayed bead-bound single cell; and a unique combination of nucleic acid-barcoded identifying particles. Image data of the indexed partitions is then obtained to identify the unique combination of nucleic acid-barcoded identifying particles for the indexed partitions.

### Nucleic Acid-Barcoded Identifying Particles

As summarized above, embodiments of the invention include indexing partitions with unique combinations of nucleic acid-barcoded identifying particles. Nucleic acid-barcoded identifying particles (which may also be referred to as Pheno-Seq particles) are solid supports, e.g., beads, that have known size and color signature, e.g., color (i.e., hue) and brightness (e.g., as provided by fluorescent emission of one or more fluorophores incorporated into the particle). Any type of solid support, e.g., as described above, may be employed as a nucleic acid-barcode identifying particle. As such, particles may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently) and a color imparting agent, e.g., one or more fluorophores, may be incorporated. A solid support may comprise a discrete particle that may be spherical (e.g., microspheres) or have a non-spherical or irregular shape, such as cubic, cuboid, pyramidal, cylindrical, conical, oblong, or disc-shaped, and the like. While the size of nucleic acid-barcoded identifying particles may vary, in some instances the size of a given particle ranges from 1 to 50 μm, such as 2 to 25 μm and including 3 to 20 μm. In some instances, the particles have a size of 3, 7, 10 or 16 μm.

As summarized above, identifying particles employed in embodiments of the invention include a color signature (collectively made up of one or more hues and/or brightness thereof). In embodiments, identifying particles include one or more color imparting agents, e.g., pigments, fluorophores, etc., where the one or more color imparting agents and amounts thereof collectively make up the color signature of the particle.

The color signature of a given identifying particle according to embodiments of the invention is provided by one or more fluorescent dyes. Where a given color signature is provided by more than one fluorescent dye, the two or more fluorescent dyes collectively make up the color signature of the identifying particle. As such, a given color signature may, in embodiments of the invention, be made up of a single fluorescent dye, or two or more fluorescent dyes, e.g., 2 to 5, such as 2 to 4, including 2 to 3, fluorescent dyes, which collectively make up the color signature of the bead. As such, the number of different fluorophores making up a given color signature may vary, ranging in some instances from 1 to 5, such as 1 to 3 and including 1 to 2. Any given two distinguishable color signatures may be distinguishable from each other based on the types of fluorophores and/or signal brightness provided thereby. As such, any two distinguishable color signatures of distinct identifying particles may be distinguishable based on fluorescent signals (e.g., emission wavelength maxima) and/or intensity thereof, of the fluorescent dyes and/or amount thereof collectively making up the color signature. For example, two distinguishable color signatures of two different identifying particles may be distinguishable from each other because they are made up of combinations of different types fluorophore dyes, e.g., where one includes fluorophore the other includes fluorophore b. Two distinguishable color signatures may also be distinguishable from each other because they are made up of different amounts of fluorescent dyes, e.g., where one is made up of fluorophore a present in a first amount present in a given identifying particle and the other is made up of fluorophore a present at a second amount that differs from the first amount at a value that can be detected, e.g., by a difference in brightness of signal.

Combinations of type and amount of fluorophores may be employed to provide any desired number of unique color signatures.

Color signatures include one or more fluorophores, as desired. As such, an identifying particle may include a single type of fluorophore. Alternatively, a given identifying particle may include two or more different types fluorophores. Examples of fluorophores that may be present in identifying particles include, but are not limited to: acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl) maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, IL), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes (Eugene, Oreg.) and Exciton (Dayton, Ohio).

In some instances, the fluorophore is a polymeric dye (e.g., fluorescent polymeric dye). Fluorescent polymeric dyes that find use in the subject methods are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where IT-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. The structural characteristics of the polymeric dyes can have an effect on the fluorescence properties of the molecules.

Any convenient polymeric dye may be utilized. In some instances, a polymeric dye is a multichromophore that has a structure capable of harvesting light to amplify the fluorescent output of a fluorophore. In some instances, the polymeric dye is capable of harvesting light and efficiently converting it to emitted light at a Ionger wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer), and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described in U.S. Pat. Nos. 7,270,956; 7,629,448; 8,158,444; 8,227,187; 8,455,613; 8,575,303; 8,802,450; 8,969,509; 9,139,869; 9,371,559; 9,547,008; 10,094,838; 10,302,648; 10,458,989; 10,641,775 and 10,962,546 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety. Specific polymeric dyes that may be employed include, but are not limited to, BD Horizon Brilliant™ Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515, BB550, BB790) (BD Biosciences, San Jose, CA). Any fluorochromes that are known to a skilled artisan-including, but not limited to, those described above- or are yet to be discovered may be employed in the subject methods.

In some instances, each of the one or more fluorophores that make up a given color signature is excitable by common light source, such as a common laser. In such instances, each of the plurality of fluorophores that make up a given color signature may have a common excitation maximum, but differ from each other in terms of emission maximum.

As reviewed above, any given two distinguishable color signatures may be distinguishable from each other based on the types of fluorophores make up the barcode and/or signal brightness provided thereby. As such, any two different color signatures may be distinguishable based on fluorescent signals and/or intensity thereof, of the fluorescent signals obtained from the color signature. For example, two distinguishable color signatures may be distinguishable from each other because they are made up of different types fluorophores, e.g., where one includes fluorophore a and the other includes fluorophore b. Two distinguishable color signatures may also be distinguishable from each other because they are made up of different amounts of fluorophores, e.g., where one is made up of fluorophore a in a first amount associated with the identifying particle and the other is made up of fluorophore a present at a second amount that differs from the first amount at a value that can be detected, e.g., by a difference in brightness of signal. Different brightness among identifying particles may readily be provided by having differing amounts of fluorophore(s) associated with the particle. Combinations of type and amount of fluorophores may be employed to provide any desired number of unique color signatures.

In addition to the color signatures, nucleic acid-barcoded identifying particles employed in embodiments of the invention include an oligonucleotide barcode component, which may be referred to as a Pheno-Seq oligonucleotide barcode component. Oligonucleotide barcode components may vary in length, ranging in some instances from 10 to 500 nt, such as 15 to 100 nt. In some instances, the oligonucleotide barcode component may be made up of ribonucleic acids or deoxyribonucleic acids, as desired. Oligonucleotide barcode components of embodiments of the invention may include an identifying particle (i.e., Pheno-Seq particle) barcode domain, as well as other domains that find use in embodiments of the invention, where such domains may include, but are not limited to, an identifying particle (i.e., Pheno-Seq particle) (i.e., Pheno-Seq particle) primer binding site, a second domain complementary to a sequence present in cell-binding bead nucleic acids, etc. The barcode component may be covalently bound directly to the particle, or via an intermediate group, e.g., a disulfide bond, as desired. In certain embodiments, the cell-binding bead nucleic acid may be linked to the bead by a cleavable linker, which linker may be cleaved by cell lysis conditions, where examples of such linkers include, but are not limited to: disulfide linkers, and the like.

An identifying particle barcode domain is a unique identifier and is a domain or region that may be employed, e.g., by its sequence, to identify the identifying particle with which it is associated. The unique identifiers can be, for example, a nucleotide sequence having any suitable length, for example, from about 4 nucleotides to about 200 nucleotides. In some embodiments, the unique identifier is a nucleotide sequence of 25 nucleotides to about 45 nucleotides in length. In some embodiments, the unique identifier can have a length that is, is about, is less than, is greater than, 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 70 nucleotides, 80 nucleotides, 90 nucleotides, 100 nucleotides, 200 nucleotides, or a range that is between any two of the above values.

Oligonucleotide barcodes of dual indexed beads may include an identifying particle primer binding site. A primer binding site, when present, may be configured to bind to a primer employed, e.g., in preparing sequence-able nucleic acids. In embodiments, the identifying particle primer binding site is employed with a primer that is distinct from any universal primer that may be employed in a given workflow. As such, the identifying particle primer binding site may bind to a primer that is configured to prime nucleic acid synthesis using only identifying particle nucleic acids as a template, and not other nucleic acids that may be present. In some instances, a primer binding site can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. An identifying particle primer binding site can vary in length, and can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. An identifying particle configured to be employed with such primer binding sites can vary in length, and in some instances can range from 5-30 nucleotides in length. The primer binding site can be positioned at the 5′ end of the oligonucleotide barcode component.

The oligonucleotide barcode may include a cell-binding bead complementary sequence, e.g., a domain or region that is complementary to a sequence present in cell-binding bead nucleic acids, such as described above. Cell-binding bead complementary sequences of interest may vary, as desired, and may be specific or random or semi random. In some instances, the cell-binding bead complementary sequence is a sequence that hybridizes to domain or region of a cell-binding bead nucleic acid, e.g., as described in greater detail above. While the length of this domain may vary, in some instances, the length of this domain ranges from 3 to 50, such as 5 to 25 nt. When present, this domain may be positioned at the 3′ end of the oligonucleotide barcode.

As reviewed in greater detail below, partitions are indexed with a unique combination of identifying particles. Among the plurality of distinct identifying particles making up a given unique combination, the oligonucleotide barcode components may share common domains. For example, the oligonucleotide barcode components of the identifying particles may have common primer binding sites, domains that are complementary to cell-binding bead nucleic acids, etc. In such instances, the common domains may have the same sequences, such that the distinct particles of the unique combination have identical common sequences, e.g., identical primer binding sites, identical cell-binding bead nucleic acid complementary domains, etc.

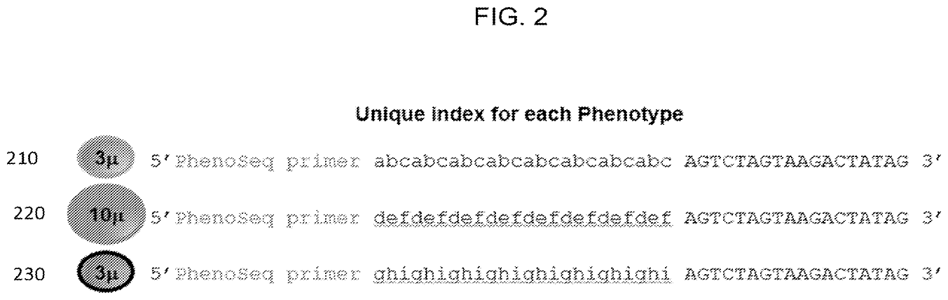

FIG. 2 provides an illustration of three distinct identifying particles, 210, 220 and 230, in accordance with an embodiment of the invention. As shown in FIG. 2, each distinct identifying particle includes a particle and an oligonucleotide barcode component. Identifying particle 210 includes 3 μm particle having a green color as well as oligonucleotide barcode component that includes an identifying particle primer binding site at the 5′ end, a unique barcode domain and a cell-binding bead complementary domain at the 3′ end. Identifying particle 220 includes 10 μm particle having a red color as well as oligonucleotide barcode component that includes an identifying particle primer binding site at the 5′ end, a unique barcode domain and a cell-binding bead complementary domain at the 3′ end. Identifying particle 230 includes 3 μm particle having a grey color as well as oligonucleotide barcode component that includes an identifying particle primer binding site at the 5′ end, a unique barcode domain and a cell-binding bead complementary domain at the 3′ end. Where desired, the barcode components may be covalently bound directly to particles or via an intermediate group, e.g., a disulfide bond, e.g., as described above. The particles may have any convenient color, where examples of suitable colors include, but are not limited to: green, red, blue, grey, yellow and black.

### Introducing Unique Combinations of Distinct Nucleic Acid-Barcoded Identifying Particles to Partitions

For visual indexing of partitions containing functionally assayed single cells, embodiments of the methods include introducing unique combinations of distinct nucleic acid-barcoded identifying particles, e.g., as described above, to partitions that include functionally assayed bead-bound single cells to produce indexed partitions, where the indexed partitions include a functionally assayed bead-bound single cell; and a unique combination of nucleic acid-barcoded identifying particles.

The unique combinations of distinct nucleic acid-barcoded identifying particles which are provided to partitions that include functionally-assayed single cells is made up of a plurality of distinct nucleic acid-barcoded identifying particles that differ from each other by one or more of size, color and brightness. The number of distinct nucleic-acid barcoded identifying particles making up a given unique combination that may be present in a given partition may vary, and in some instances ranges from 1 to 15, such as 1 to 10 and including 1 to 5, such as 2 to 5. In a given unique combination, the size of the different identifying particles may vary, ranging in some instances from 1 to 50 μm, such as 2 to 25 μm and including 3 to 20 μm. In some instances, the particles making up a given unique combination have a size of 3, 7, 10 or 16 μm. The colors of the different identifying particles making up a given unique combination (e.g., as provided by fluorophores of the identifying particles) may also vary, where in some instances colors of different particles making up a unique combination may be green, red, blue, grey, black or yellow. Brightness may also vary among particles making up a unique combination, e.g., where at least one of the particles is bright and at least one of the particles is dim.

Embodiments of the methods including introducing unique combinations of identifying particles to partitions that include functionally assayed single cells in order to provide a visual index for those partitions, where the visual index is provided by the unique combination of identifying particles present in the partition. The unique combination of identifying particles may be introduced into partitions using any convenient protocol. In some instances, introducing unique combinations of distinct nucleic acid-barcoded identifying particles to partitions that include functionally-assayed bead-bound single cells includes introducing a composition of distinct nucleic acid-barcoded identifying particles into a flow cell having microwells on a bottom surface thereof, wherein the microwells include functionally-assayed bead-bound single cells.

Compositions of nucleic-acid barcoded identifying particles employed in embodiments of the invention may be compositions made of a plurality of distinct nucleic-acid barcoded identifying particles present in a liquid, e.g., aqueous liquid. The composition can be contacted with compartments, for example, by gravity flow wherein identifying particles can settle into the partitioning structures. In some instances, an aqueous composition of the identifying particles is contacted with, e.g., by flowing it across, an array of microwells such that identifying particles are deposited into the microwells. The aqueous composition that includes the identifying particles may be flowed through a flow cell in fluidic communication with the microwells. Suitable protocols and systems for partitioning the bead-bound cells into microwells are described in PCT application serial no. PCT/US2016/014612 published as WO/2016/118915, the disclosure of which is herein incorporated by reference. In order to ensure a sufficient number (e.g., as described above) of identifying particles are deposited into partitions, embodiments of the methods may further include introducing a second composition of distinct nucleic acid-barcoded identifying particles into the flow cell such that particles are deposited into partitions. The number of such iterations may vary as desired, ranging in some instances from 2 to 5, such as 2 to 4, and including 2 to 3 iterations.

### Obtaining Image Data of Indexed Partitions

Following introduction of unique combinations of identifying particles into partitions to produce indexed partitions, e.g., as described above, embodiments of methods of the invention include obtaining image data of the indexed partitions to identify the unique combination of nucleic acid-barcoded identifying particles therein, and thereby obtain indexing data for the partitions. The indexed partitions may be imaged using any convenient protocol to obtain image data of the partitioned single cells. Image data that is obtained may vary. Image data may be obtained for any partitioned cell of interest, and may be obtained from partitions containing functionally assayed cells of interest. The type of image data that is obtained may vary, and may include live cell image data. Any convenient protocol may be employed to obtain image data for cells in partitions, where examples of imaging protocols that may be employed include, but are not limited to, microscopic imaging protocols, such as phase contrast microscopy, fluorescence microscopy, quantitative phase-contrast microscopy, holotomography, BD Rhapsody System (Becton, Dickinson and Company), and the like. An image may be generated by, for example, fluorescent imaging. Imaging can include microscopy such as bright field imaging, oblique illumination, dark field imaging, dispersion staining, phase contrast, differential interference contrast, interference reflection microscopy, fluorescence, confocal, and single plane illumination, or any combination thereof. Imaging can include imaging a portion of the sample (e.g., slide/array). Imaging can include imaging at least 10, 20, 30, 40, 50, 60, 70, 80, 90, or 100% of the partitioned cells. In some instances, imaging can be done in discrete steps (e.g., the image may not need to be contiguous). Imaging can include taking at least 1, 2, 3, 4, 5, 6, 7, 8, 9, or 10 or more different images. Imaging can include taking at most 1, 2, 3, 4, 5, 6, 7, 8, 9, or 10 or more different images.

In embodiments of the invention, obtaining image data for the partitioned single cells includes obtaining a partition specific visual index for the partitions of interest and therefore the functionally-assayed, bead-bound cells present therein. As such, methods may include, for each partitioned, functionally-assayed, bead-bound cell of interest, obtaining a partition specific identification of the unique combination of identifying particles present therein, and therefore an identification of the combination of identifying particles that are present in the same partitions as that cell. The visual index may be viewed as the image data of the unique collection of identifying particles present in a given partition.

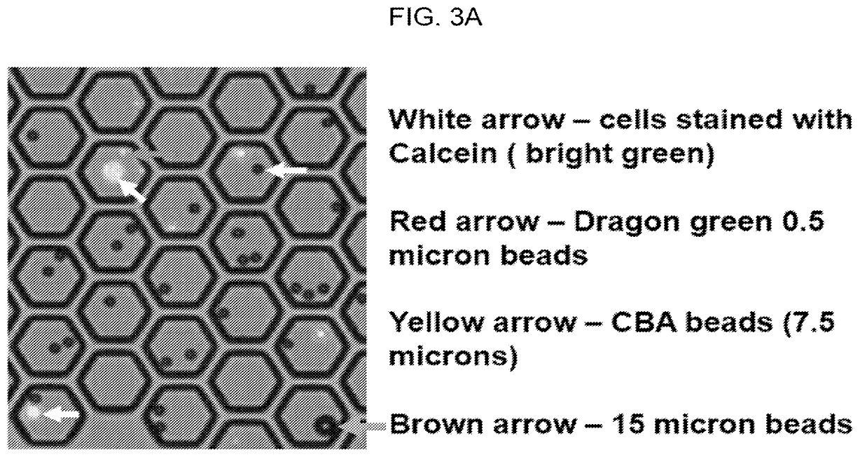

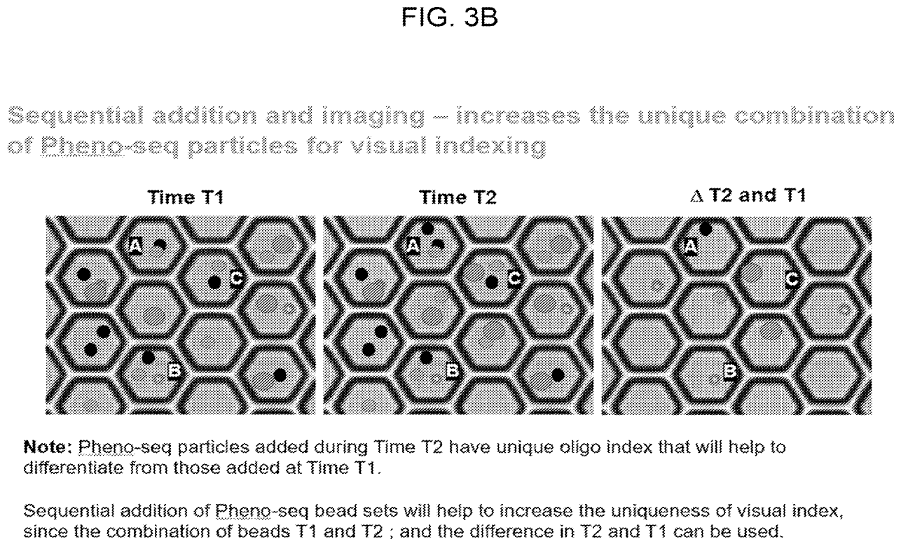

FIG. 3A provides shows cell containing partitions visually indexed with unique combinations of identifying particles. As can be seen in FIG. 3, different wells have unique combinations of identifying particles, the image of which can be used to visually index those wells. As shown, two different wells contain cells (white arrows). The first of these wells also includes two 0.5 micron identifying particles, providing a visual index for that well. The second of these wells includes 7.5 micron black bead, providing a visual index for that well. The visual index of the first well is distinct from the visual index for the second well. As described in greater detail below, sequence reads obtained from the identifying particle barcode components can be employed, in combination with the visual index, to determine the wells from which those reads were obtained. The reads can thus be matched with any functional data obtained for those wells. By then matching the reads with cellular target reads, as described in greater detail below, the functional data can be linked with sequence data, e.g., omic data, for the wells. In some embodiments, a sequential imaging protocol is employed to visually index wells. By sequentially imaging, lower numbers and/or combinations of pheno-seq particles can be employed to visually index more wells, e.g., as compared to a protocol that is not sequence, e.g., as illustrated in FIG. 3A. In sequential imaging, two or more pheno-seq particle compositions are sequentially introduced into partitions, with images obtained following each introduction. In other words, two more iterations of pheno-seq particle partitioning followed by imaging are conducted. while the number of iterations may vary, in some instances the number of iterations ranges from 2 to 5, such as 2 to 4, e.g., 2 to 3. At each time point following partitioning, the partitioned pheno-seq beads will have unique indexed oligos. An embodiment of a sequential imaging protocol to visually index partitions is illustrated in FIG. 3B. As illustrated in FIG. 3B, at Time T1: Wells A, B, C have the same combination of black and green beads. However, when pheno-seq particles/beads are then added at T2 time point: Well “A” gets additional black, while Well “B” gets none and Well “C” gets red particle. It is noted that the pheno-seq particles added in T2 have different oligo sequence and can be differentiated by sequencing results, e.g., as described in detail below. The difference of Image at T1 and T2 is denoted by the A T1 and T2 image. As illustrated, even though the same black and red particles are present in T2, the oligos are different and since its differentiated by time points, one can visually index more wells/cells, e.g., as compared to protocols where only single partitioning/imaging steps are employed, e.g., as illustrated in FIG. 3A.

### Obtaining Sequence Data

Following production of visually-indexed, functionally-assayed, partitioned single cells, e.g., as described above, embodiments of methods may include obtaining sequence data for the visually-indexed, functionally-assayed, partitioned single cells. Sequence data may be obtained for the visually-indexed, functionally-assayed, partitioned single cells using any convenient protocol. In some instances, the sequence data is obtained using a protocol that produces a sequenceable library of nucleic acids for visually-indexed, functionally-assayed, partitioned single cells, followed by sequencing that library, e.g., by using a next generating sequencing protocol.

### Production of Sequenceable Library

Sequenceable libraries of nucleic acids can be prepared from partitioned cells using any convenient protocol. Of interest are protocols that produce libraries which include a cell label domain, which domain can be used to identify nucleic acids as being derived from a given partitioned single cell, and distinguish nucleic acids derived from other partitioned single cells. In some instances, the protocol used to prepare sequenceable libraries is a cell capture bead mediated protocol. In such instances, functionally-assayed, bead-bound, partitioned single cells may be in spatial proximity to a cell capture bead, having bound cell label domain nucleic acids that include a target binding region, e.g., as described in greater detail below. When cell label domain nucleic acids are in close proximity to targets of the functionally assayed, partitioned single cells, the targets can hybridize to the cell label domain nucleic acid. The cell label domain comprising nucleic acid can be contacted at a non-depletable ratio such that each distinct target can associate with a distinct cell label domain comprising nucleic acid having its own unique UMI, if so desired.

As such, in some embodiments, the methods further include providing a cell capture bead that includes a cell label containing bead bound nucleic acid into partitions that include the functionally-assayed single cells, where the cell label containing nucleic acid is employed in preparing nucleic acid sequence ready compositions, e.g., sequence ready libraries, from the functionally-assayed, partitioned single cells. The cell capture bead nucleic acid includes a cell label. A cell label is a unique identifier and is a domain or region that may be employed, e.g., by its sequence, to identify the cell capture bead with which it is associated, as well as cell in proximity thereto. The unique identifiers can be, for example, a nucleotide sequence having any suitable length, for example, from about 4 nucleotides to about 200 nucleotides. In some embodiments, the unique identifier is a nucleotide sequence of 25 nucleotides to about 45 nucleotides in length. In some embodiments, the unique identifier can have a length that is, is about, is less than, is greater than, 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 70 nucleotides, 80 nucleotides, 90 nucleotides, 100 nucleotides, 200 nucleotides, or a range that is between any two of the above values.

In some instances, the cell capture bead bound nucleic acid includes a target binding region, e.g., that binds to complementary sequences in nucleic acid species of interest in the functionally-assayed cell, as well as to capture sequences of the barcode components of nucleic acid-barcoded, cell-binding beads, e.g., as described above. For example, where target nucleic acids species are cellular mRNA and the barcode components include a poly(A) capture sequence, a cell capture bead bound nucleic acid may include a poly(T) domain as a target binding region.

In addition to the target binding region, the bound nucleic acid many further include one or more additional domains, such as but not limited to: additional barcode domains, molecular index domains (e.g., unique molecular identifier (UMI) domain), universal primer binding domains, etc. Further details regarding particles having bound nucleic acids that may be provided in compartments may be found in in U.S. Patent Application Publication No. US2018/0088112; US Patent Application Publication No. 2018/0200710; U.S. Patent Application Publication No. US2018/0346970; U.S Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference. Beads having bound nucleic acids may be provided in the compartments using any convenient protocol, including but not limited to, those described above for partitioning of cells, and further described in further described in PCT application serial no. PCT/US2016/014612 published as WO/2016/118915, the disclosure of which is herein incorporated by reference. The particles, e.g., beads, may be co-located, e.g., provided in proximity to, the cells before or after, or in some instances in combination with, the single cells, as desired.

Following production of visually-indexed, functionally-assayed, partitioned single cells in proximity to cell label domain comprising beads, e.g., as described above, the cells can be lysed to liberate the target molecules so that the released target molecules, e.g., nucleic acids, can bind to the target binding regions of the cell label domain nucleic acids to produce captured nucleic acids. Cell lysis can be accomplished by any of a variety of means, for example, by chemical or biochemical means, by osmotic shock, or by means of thermal lysis, mechanical lysis, or optical lysis. Particles can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof. To increase the association of a target and a barcode, the rate of the diffusion of the target molecules can be altered by for example, reducing the temperature and/or increasing the viscosity of the lysate. In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate. In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, 10, or more. The pH of the lysis buffer can be at most about 1, 2, 3, 4, 5, 6, 7, 8, 9, 10, or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7%, or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCI, about pH 7.5, about 0.5M LiCl, about 1% lithium dodecyl sulfate, about 10 mM EDTA, and about 5 mM DTT. Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

Following lysis of the cells and release of nucleic acid molecules therefrom, the nucleic acid molecules can randomly associate with the cell label domain nucleic acids of the co-localized cell capture bead. Association can comprise hybridization of a cell label domain nucleic acid's target recognition region to a complementary portion of the target nucleic acid molecule (e.g., oligo (dT) of the barcode can interact with a poly(A) tail of a target). The assay conditions used for hybridization (e.g., buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo (dT), mRNA molecules can hybridize to the probes and be reverse transcribed. The oligo (dT) portion of the oligonucleotide can act as a primer for first strand synthesis of the cDNA molecule, e.g., when subject to DNA synthesis reaction conditions to produce first strand cDNA domain comprising capture nucleic acids.

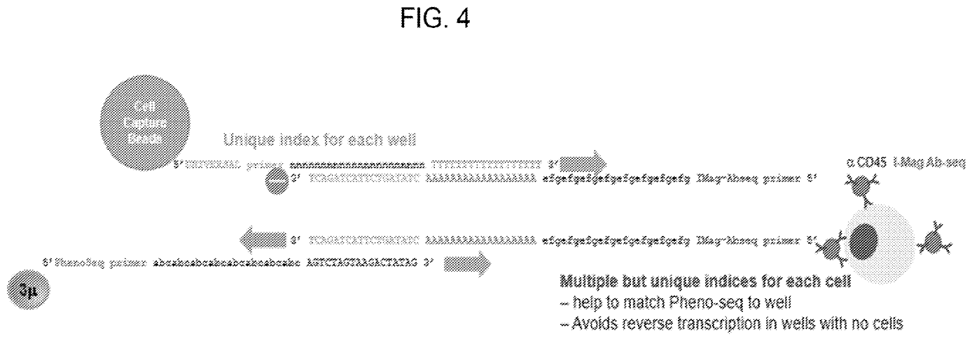

Cell label domain nucleic acid can also hybridize to complementary capture sequences of barcode components, e.g., poly(A) sequences, of the cell-binding beads stably associated with the cells. In this way, the cell label domain nucleic acids can act as primers for reverse transcription using the barcode as a template, e.g., as described in greater detail below. In addition to the production of cell-label domain/cell-binding bead nucleic acid hybridization complexes, hybridization complexes of cell-binding bead nucleic acids and identifying particle nucleic acids are also produced, as mediated by the complementary domains of the identifying particle nucleic acids and the cell-binding bead nucleic acids. FIG. 4 provides a schematic representation of the interaction of oligos between cell capture beads, cell-binding beads (i.e., IMag beads bound to cells) and identifying particles (Pheno Seq particles) in a single well. As illustrated in FIG. 4, the cell capture bead nucleic acid that includes a universal primer binding site, a cell label domain (i.e., unique index for each well) and a target binding region (polyT) hybridizes to the capture sequence (polyA) of the cell-binding bead nucleic acid. In addition, the cell-binding bead nucleic acid hybridizes to complementary domains in the identifying particle nucleic acids. In this way, reverse transcribable hybridization complexes are produce between: (1) the cell capture bead/cell-binding bead nucleic acids; and (2) the cell-binding bead and identifying particle nucleic acids.

In some instances, the methods further include employing oligonucleotide labeled cellular component binding reagents, e.g., in applications where detection, e.g., quantitation, of one of or more cellular components, e.g., surface proteins, is desired, e.g., in AbSeq applications. Oligonucleotide labeled cellular component-binding reagents employed in such embodiments include a cellular component-binding reagent, e.g., antibody or binding fragment thereof, coupled to a cellular component-binding reagent specific oligonucleotide comprising an identifier sequence for the cellular component-binding reagent that the cellular component-binding reagent specific oligonucleotide is associated therewith. In such instances, the cell capture bead may include a nucleic acid configured to capture, e.g., specifically bind to, a domain of the cellular component-binding reagent specific oligonucleotide, e.g., a polyT sequence, such as described above. In this way, protein expression may be assayed in conjunction with gene expression, e.g., where multi-omic analysis is desired, e.g., combined analysis of transcriptome and proteome. In such instances, the methods may include preparing the captured sample with oligonucleotide labeled cellular component binding reagents, and then provide for capture of cellular component-binding reagent specific oligonucleotides released from the capture, partitioned cells. Further details regarding use of oligonucleotide labeled cellular component-binding reagents are found in United States Published Patent Application Nos. US20180267036 and US20200248263; the disclosures of which are herein incorporated by reference.

Where desired, a given workflow may include a pooling step where a product composition, e.g., made up of captured nucleic acids, synthesized first strand cDNAs or synthesized double stranded cDNAs, is combined or pooled with product compositions obtained from one or more additional samples, e.g., combinatorial barcoded cells. In some instances, the pooling step is performed just after hybridization step between cell label domain nucleic acids and target nucleic acids, e.g., as reviewed above. The number of different product compositions produced from different samples, e.g., cells, that are combined or pooled in such embodiments may vary, where the number ranges in some instances from 2 to 1,000,000, such as 3 to 200,000, including 4 to 100,000 such as 5 to 50,000, where in some instances the number ranges from 100 to 10,000, such as 1,000 to 5,000. Prior to or after pooling, the product composition(s) can be amplified, e.g., by polymerase chain reaction (PCR), such as described in greater detail below. Once the target-cell domain label complexes and cell-binding bead nucleic acid/identifying particle nucleic acid complexes have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the hybridized complexes from a plurality of cells.

The disclosure provides for a method to create a target-cell label domain conjugate using any convenient protocol, such as reverse transcription or nucleotide extension. The target-cell label domain conjugate can comprise the cell label domain and a complementary sequence of all or a portion of the target nucleic acid. Reverse transcription of the associated RNA molecule can occur by the addition of a reverse transcription primer along with the reverse transcriptase. The reverse transcription primer can be an oligo (dT) primer, a random hexanucleotide primer, or a target-specific oligonucleotide primer. Oligo (dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest. Reverse transcription can occur repeatedly to produce multiple cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

One or more nucleic acid amplification reactions can be performed to create multiple copies of the target nucleic acid molecules. Amplification can be performed in a multiplexed manner, wherein multiple target nucleic acid sequences are amplified simultaneously. The amplification reaction can be used to add sequencing adapters to the nucleic acid molecules. The amplification reactions can comprise amplifying at least a portion of a sample label, if present. The amplification reactions can comprise amplifying at least a portion of the cellular label and/or barcode sequence (e.g., a molecular label). The amplification reactions can comprise amplifying at least a portion of a sample tag, a cell label, a spatial label, a barcode sequence (e.g., a molecular label), a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a barcode sequence (e.g., a molecular label).

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a QB replicase (QB) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the nucleic acid (e.g., RNA, DNA, cDNA) to produce a labeled amplicon (e.g., a stochastically labeled amplicon). The labeled amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a barcode sequence (e.g., a molecular label). The labeled amplicon can be a single-stranded molecule. The single-stranded molecule can comprise DNA, RNA, or a combination thereof. The nucleic acids of the disclosure can comprise synthetic or altered nucleic acids. As such, methods may include producing an amplicon composition from the first strand cDNA domain comprising capture nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of labeled targets (e.g., stochastically labeled targets). The one or more primers can anneal to the 3′ end or 5′ end of the plurality of labeled targets. The one or more primers can anneal to an internal region of the plurality of labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a barcode sequence (e.g., a molecular label), a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the cell label and barcode sequence (e.g., molecular label) on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonuclease digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

In some embodiments, amplification can be performed on the substrate, for example, with bridge amplification. cDNAs can be homopolymer tailed in order to generate a compatible end for bridge amplification using oligo (dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges. The amplification reactions can comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

In some embodiments, amplification of the labeled nucleic acids comprises non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a QB replicase (QB), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about 1, 2, 3, 4, 5, 6, 7, 8, 9, 10, 11, 12, 13, 14, 15, 16, 17, 18, 19, or 20 amplification reactions. Alternatively, the method comprises conducting at least about 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids, the internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, barcoding (e.g., stochastically barcoding) the plurality of targets in the sample further comprises generating an indexed library of the barcoded targets (e.g., stochastically barcoded targets) or barcoded fragments of the targets. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adapters can be added for sequencing using, for example, next generation sequencing (NGS). The sequencing results can be used to determine cell labels, molecular labels, and sequences of nucleotide fragments of the one or more copies of the targets.

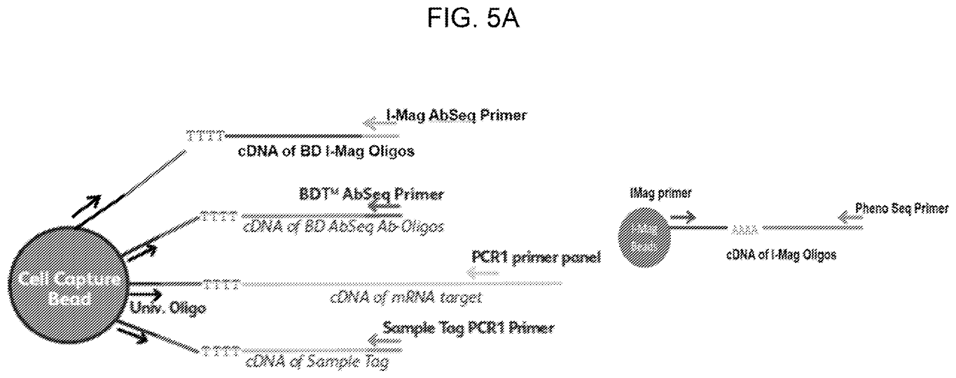

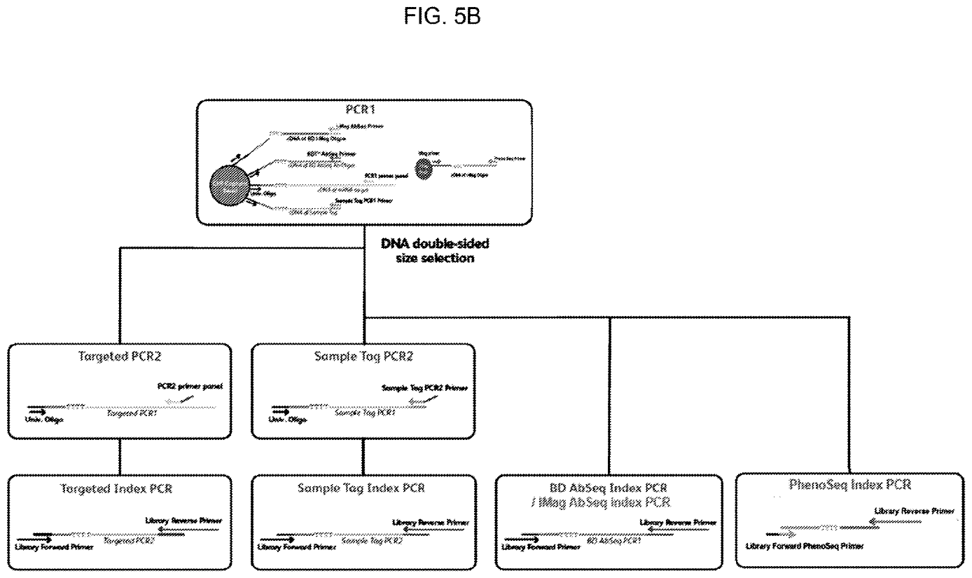

In certain embodiments, the methods provided further include subjecting a prepared expression library, e.g., an amplicon composition produced as described above, to a sequencing protocol, such as an NGS protocol. The protocol may be carried out on any suitable NGS sequencing platform. NGS sequencing platforms of interest include, but are not limited to, a sequencing platform provided by Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLID sequencing system); Oxford Nanopore (e.g., Minion), Roche (e.g., the 454 GS FLX+ and/or GS Junior sequencing systems); or any other sequencing platform of interest. The NGS protocol will vary depending on the particular NGS sequencing system employed. Detailed protocols for sequencing, e.g., which may include further amplification (e.g., solid-phase amplification), sequencing the amplicons, and analyzing the sequencing data are available from the manufacturer of the NGS sequencing system employed. FIGS. 5A and 5B provide an illustration of an amplification workflow that may be employed to produce a sequence ready library in accordance with embodiments of the invention. FIG. 5A illustrates how cell-binding bead primers (I-Mag AbSeq primers) are included in amplifications of the signal from cell-binding beads (IMag AbSeq beads) and (C) how cell-binding bead primers (I-Mag AbSeq primers) and identifying particle primrs (Pheno Seq primers) are included in amplifications of the signal from cell-binding beads (IMag AbSeq beads) and identifying particles (Pheno Seq) particles. FIG. 5B illustrates a workflow that includes the final step of including library sequences for identifying particle (PhenoSeq) oligo products during an identifying particle (PhenoSeq Index) PCR step. This step includes a modified PCR forward primer which is a combination of a Library Forward primer and PhenoSeq primer sequence, which is employed since the identifying particle/cell-binding bead (e.g., PhenoSeq-IMag AbSeq) product amplified from the cell-binding bead nucleic acids (IMag bead nucleic acids) lacks a Universal primer sequence.

Further details regarding methods for obtaining sequence data from single cells, e.g., as described above, are provided in U.S. Patent Application Publication No. US2018/0088112; US Patent Application Publication No. 2018/0200710; U.S. Patent Application Publication No. US2018/0346970; U.S Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

The sequencing protocol generates sequence data for the combinatorial barcoded cells. This sequence data can then be readily linked to image data for the combinatorial barcoded cells, such that image data and sequence data obtained from the same combinatorial barcoded cells may be paired. In other words, a given set of image data and a given set of sequence data may be linked as being obtained from the same combinatorial barcoded cell, e.g., as described in greater detail below.

### Linking Single-Cell Functional and Sequence Data

Following obtainment of sequence data, e.g., as described above, embodiments of the methods include linking functional and sequence data for sequenced, visually-indexed, functionally-assayed, partitioned single cells. In such instances, the obtained functional and sequence data obtained from a given partition, and therefore a cell present in that partition, is linked. By “linked” is meant that functional and sequence data are paired as originating from the same partition, and therefore cell that was present in that partition when the functional data for that partition was obtained. As such, functional data and sequence data obtained from the same cell may be paired. In other words, a given set of functional data and a given set of sequence data, which may be omic data, such as transcriptome and proteome data (such as obtained with an AbSeq platform) may be identified as being obtained from the same cell and then paired or otherwise associated with each other. In this manner, linked functional and sequence data may be obtained for single cells of a cellular sample.

The functional data and sequence data is linked by using the cell-binding bead nucleic acid barcodes to identify sequence reads of nucleic acid amplicons that are obtained from the same partition. In the obtained sequence data, e.g., as described above, sequence reads for cellular targets, cell-binding bead nucleic acids and identifying particle nucleic acids are obtained. Reads will be obtained from nucleic acids that include both the cell-binding bead nucleic acid barcode and the cell label barcode, which cell label barcode is also present in all reads of cellular target nucleic acids present in a given partition. In addition, reads will be obtained from nucleic acids that include both the cell-binding bead nucleic acid barcode and the identifying particle barcode provided by identifying particles from a given partition. The cell-binding bead nucleic acid barcode may be used to identify the partition from which reads arising from cellular targets and reads arising from identifying particles are obtained. In other words, for each cell assayed in a given workflow, the sequences of identifying particle barcodes associated with a partition containing that cell and the sequence of target nucleic acids from that cell, e.g., mRNAs from the cell, are obtained. For each cell, these obtained sequences are obtained using a protocol (which may be a next generation sequencing protocol), such as described above, where a library is generated from the original sequences, where each member of a given library generated from the same partition shares a common cell label for cellular targets and identifying particle nucleic acids that can be linked by a common cell-binding bead nucleic acid barcode. As such, sequence reads from the cellular target nucleic acids and the identifying particles that are obtained from the same cell can be identified as coming from the same partition by use of the cell-binding bead nucleic acid barcode to associate the disparate reads. In linking the functional and sequence data, all reads that have the same cell label domain, i.e., that share a common cell label, from both: (a) reads of target nucleic acids; and (b) reads of cell-binding bead nucleic acids, may be paired or linked. This pairing or linkage results in a set of reads that includes reads of target nucleic acids and cell-binding bead barcode nucleic acids, and these reads can be identified as originating from the same cell. In addition, all reads that have the same cell-binding bead nucleic acid barcode, i.e., that share a common cell-binding bead nucleic acid barcode, from both reads of identifying particle nucleic acids and reads of cell-binding bead nucleic acids, may be paired or linked. The commonality of the cell-binding bead nucleic acid barcode allows one to pair or link the reads from cellular targets and reads from identifying particles, allowing one to identify such reads as arising from the same partition and therefore cell present therein.

Next, the resultant sequence data that includes reads of both target nucleic acids and identifying particles may be matched, i.e., paired or linked, with functional data. As reviewed above, functional data for cells may be assigned to a particular partition, which partition is visually indexed by the unique combination of identifying particles present in that partition. Reads from the unique combination of identifying particles are obtained for a given partition, e.g., as described above, and then determined to be obtained from that partition by matching or linking the reads with the visual index obtained for that partition. Different partitions of a given workflow will have their own unique visual index provided by the unique combination of identifying particles present therein. A given unique combination of identifying particles making up such a partition specific visual index can be assigned to a given portion of a sequence read because the sequences of barcodes of identifying particles from which that visual index is obtained are known. As such, each partition specific visual index obtained for a given partition and cell that is present in that partition can be used to determine the sequences of the different identifying particle barcodes associated with that cell. As the sequences of the identifying particle barcodes are present in the reads of the barcodes, a given set of identifying barcodes may be determined as being associated with a given set of sequence data. Once a partition specific set of identifying particle barcode sequence reads is associated with the given set of sequence data, the sequence data can be determined as being obtained from the same cell that was in that partition from which the set of identifying particle sequence reads was obtained. As such, from the visual index (that is a composite of the different signals obtained from the unique combination of identifying particles in a partition) obtained from a given partition, a series of sequences of identifying particle barcode regions may be obtained for that given partition. This series or collection of sequences of identifying particle barcode regions may then be used to identify all sequence data obtained from that partition, e.g., by using the cell-binding bead nucleic acid barcode to link the sequences, e.g., as described above. This identification may be done by determining that sequence reads having: (a) a common cell barcode and a cell-binding bead nucleic acid barcode; and (b) the partition identifying collection of sequences of identifying particle barcodes that also include the cellObinding bead nucleic acid barcode; are obtained from a cell that was present in a partition. Once the sequence data is assigned to a given partition, the sequence data may then be readily linked with functional data obtained from that partition. In this manner, linked functional and sequence data may be obtained for single cells of a cellular sample.

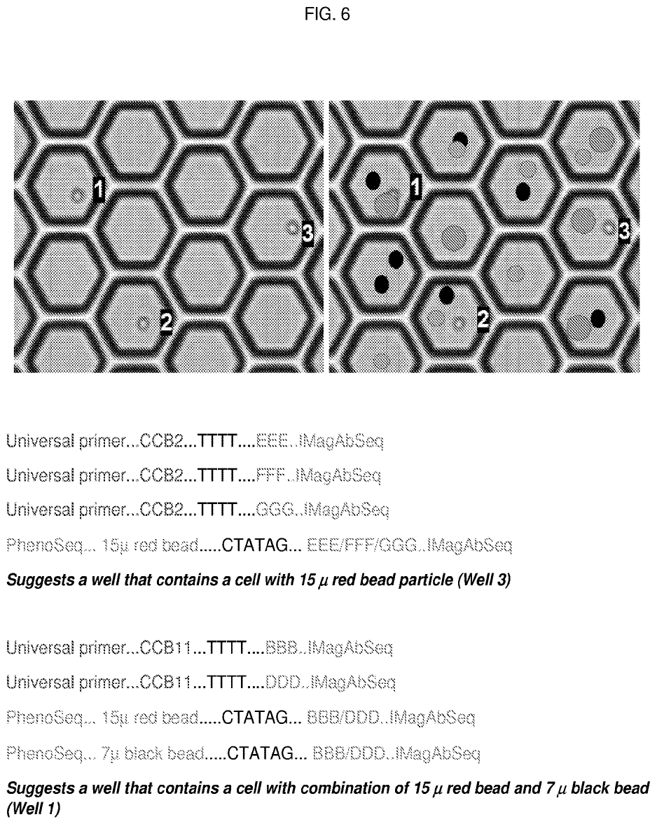

FIG. 6 provides an illustration of how to bioinformatically deconvolute the identity of the single cells and correlate with their function/phenotype. As illustrated, each well is associated with a unique index sequence derived from a Cell Capture Bead (CCB #) present in that well, multiple but unique index sequences derived from cell-binding bead (IMag AbSeq beads) (BBB, DDD, . . . . GGG) and unique sequence that are present on identifying (Phenoseq) particles (15μ red bead, 7μ black bead, 7μ green bead).

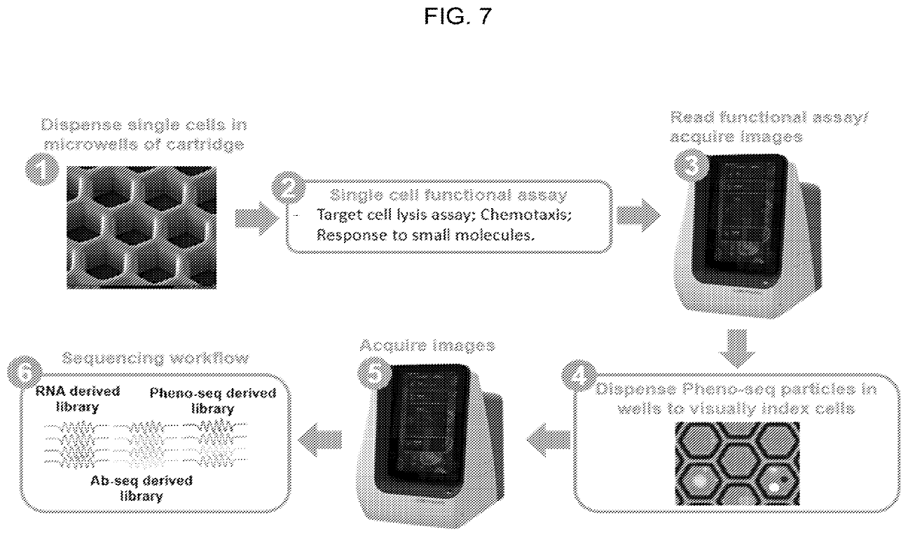

Representative Workflow A representative workflow according to an embodiment of the invention is illustrated in FIG. 7. In FIG. 7, a workflow that is performed on the BD Rhapsody™ system is shown. As illustrated, the BD Rhapsody™ is employed as a platform to evaluate single cell phenotype and function in real time and combine the resultant data with CITE-Seq (Cellular indexing of Transcriptome, Epitopes) information. As shown, the BD Rhapsody™ scanner and cartridge support single cell functional assays such as—Target cell lysis assays, chemotaxis assays and assays to evaluate response to small molecules. Images obtained from steps 3 and 6 are employed to visually index cells by Pheno-seq particle combination and match with associated phenotype, function. (The cell capture bead associated index is common for the transcriptome and epitope information associated with each single cell and is also matching with the PCR product amplified from oligos tagged to Pheno-seq particles. The information together is employed to combine cellular phenotype, function with single cell transcriptome and epitope data.)

The workflow for proposed methodology is as follows:
1. Mix cells obtained from in vitro culture or ex-vivo with “iMag AbSeq” beads (iMag beads with Antibodies targeting cells of interest such as CD45⁺ leukocytes)
2. The cells with bound iMag AbSeq beads are dispensed in Rhapsody cartridge to obtain single cells in well
3. Perform single cell functional assay in the wells of the Rhapsody cartridge
4. Read single cell functional assay output and capture cell phenotype by Rhapsody scanner.
5. Optional step: Wash off reagents, (additional cells-to mimic cancer microenvironment, target cells) from functional assay, while the cartridge is on magnet. IMag AbSeq beads bound to cells of interest help to retain them in the wells.
6. Add Pheno-seq particles to achieve a mean of 6 particles per well. Image the wells and verify if the majority of the wells with single cells are indexed with a unique combination of Pheno-seq particles. More Pheno-seq particles can be included sequentially, untill most of the wells with single cells are indexed
7. Dispense Cell capture beads and continue with current Rhapsody workflow to lyse cells and subsequent steps.
8. Additional libraries are included to amplify PCR products from oligos bound to IMag Ab-seq beads
9. Sequencing result is correlated with Rhapsody scanner images to identify associated phenotype and function of single cells

### Kits

Aspects of the invention further include kits and compositions that find use in practicing various embodiments of methods of the invention. Kits of the invention may include one or more of: a population cell-binding beads and/or cell-binding bead nucleic acid primers; a population of distinct identifying particles and/or identifying particle nucleic acid primers; beads comprising a bead bound nucleic acid comprising a cell label domain and target binding region, e.g., as described above, etc.

The kits may further include one or more additional components finding use in practicing embodiments of the methods. For example, the kits may include one or more components employed in obtaining sequence data, e.g., one or more of: primers, a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), a stimulus response polymer, or any other desired kit component(s), such as devices, e.g., as described above, solid supports, containers, cartridges, e.g., tubes, beads, plates, microfluidic chips, etc. Components of the kits may be present in separate containers, or multiple components may be present in a single container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

The following is offered by way of illustration and not by way of limitation.

### EXPERIMENTAL

### 1. Pheno Seq Particles

Pheno-seq particles (also referred to herein as identifying particles) are a combination of numerous micron size particles that have unique phenotypes (size, color, shape or fluorescence intensity) and are tagged with oligonucleotides (like Ab-seq antibodies). Each unique Pheno-seq particle is associated with a unique oligonucleotide sequence that can be matched to the particle. The combination of Pheno-seq particles is used to index different cells in a Rhapsody cartridge. A combination of 60 Pheno-seq particles, added sequentially, with a target of 6 or less in a well, is able to index more than 300 million wells with cells. Current workflow on Rhapsody supports 5000-10000 single cells on a cartridge. To index ~10000 wells with single cells, 20 Pheno-seq particles, with a target of 6 or less in a well is sufficient. The unique oligonucleotide sequences tagged to these particles helps to produce a set of unique indexed PCR products when combined with cell capture beads. Since the sequence of oligos tagged with each Pheno-seq particle is unique, the sequencing data can be deconvoluted and matched with the combination of Pheno-seq particles in a well.

### II. Visual Indexing

Here, we describe a method to transform each single cell location in the wells that contain a cell, to a unique bar code index that can be identified by the sequencing data obtained by Rhapsody workflow. Particles that can be differentiated based on their size, shape and color are tagged with unique sequences like Ab-seq oligos with some minor changes in the design of tagged oligonucleotides.

As demonstrated in Table 1, particles are dispensed at random and follow Poisson distribution. If we start with a pool of 20 unique phenotype particles and aim to get a mean of 6 particles per well, median 50% of the wells contain 4-7 particles and can index ~39000 wells with cells. It is highly likely that most of the wells that contain the single cells (2.5-5%) have a unique combination of Pheno-seq particles. It is noted that unique combination of beads in the well that contain the single cell is important (even if same combination is present in the well that does not contain a cell, the information is not conflicting as this product is not amplified).

TABLE 1
Cumulative Poisson distribution of Pheno-seq particles
with target mean of 6 particles per well, with
20 different Pheno-seq particles in the pool.

| | | | Possible |
|---|---|---|---|
| | Cumulative | Number | combinations |
| Number | Distribution | of wells | (objects = 20) |
| 0 | 0.002 | 496 | 1 |
| 1 | 0.017 | 3470 | 20 |
| 2 | 0.062 | 12394 | 190 |
| 3 | 0.151 | 30241 | 1140 |
| 4 | 0.285 | 57011 | 4845 |
| 5 | 0.446 | 89136 | 15504 |
| 6 | 0.606 | 121261 | 38760 |
| 7 | 0.744 | 148796 | 77520 |
| 8 | 0.847 | 169447 | 125970 |
| 9 | 0.916 | 183215 | 167960 |
| 10 | 0.957 | 191476 | 184756 |
| 11 | 0.980 | 195982 | 167960 |

If the number of single cell containing wells are not well discriminated from each other, then one can sequentially include additional particles in multiple batches of 20 particles with unique index sequence with a target Mean number of 8 that can be differentiated in images captured by the Rhapsody Imager. Results from sequential inclusion of cells or different size beads indicate that the Rhapsody cartridge supports to include cells and micron sized particles in sequential manner that can be imaged by the scanner multiple times.

To obtain 20 beads a combination of size (3, 7, 10, 16 microns spherical beads), fluorescence intensity (none, two different intensities (dim/bright) of green, red and double positive red and green, at least 5 that can be detected clearly by scanner). Total=4 (size)×5 (combination of fluorescence)=20 beads

The oligonucleotide tagged to the Pheno-seq particles are designed specifically to amplify only when the well contains the cells but not from the wells that do not contain the cells. To achieve this, the cells will be tagged with beads like I-Mag beads but will also include unique indexed oligonucleotide. These beads have oligonucleotides that have unique index but are conjugated to beads either with or without disulphide bond. These oligos will have common IMag-AbSeq primer region, followed by unique index sequence, following by Poly A to base pair with poly T in Cell Capture Bead oligos, and a common sequence that is complementary to 3′ end of the Pheno-Seq oligos.

### III. Technical Effect

We expand the current features of Rhapsody cartridge and scanner as a platform to perform single cell functional assay. We identify the genotype, transcriptome and epitope information associated with cell function and phenotype. Each Rhapsody cartridge has ~200,000 wells and current Rhapsody workflow recommends to include 5000-10000 single cells per cartridge. This represents 2.5-5% of all the wells. Here, we transform these 5000-10000 single cell locations in the wells to a unique bar code index that can be identified by the sequencing data obtained by Rhapsody workflow. This when combined with CITE-seq data for each single cell will identify genotype, transcriptome and associated epitope information for cell function and phenotype at single cell level.

Though combining protein expression data along with transcriptome data contributed to the robustness of the information obtained from single cells, multiple genes, post transcriptional and post translational factors, signaling pathways also regulate cell function. Understanding the omics data in relation to cellular function and phenotype is valuable to further our understanding and develop better strategies in translational research, including identifying novel biomarkers, developing diagnostic assays and exploring novel therapeutics and clinical solutions.

Tumor associated immune cells such as CD8 T cells is a good example, where these cells share transcriptome and epitope information but can vary in their function of polyfunctionality (degree and breath of cytokine secretion), proliferation capacity and effectiveness in lysing target cells. Subsets of CD8 T cells have very distinct functions such as cell killing or immune regulation. CD8+Polyfunctional T cells (which secrete more than 2 cytokines) are more effective and associated with better outcomes in immune control of cancer and infectious diseases. TCR sequence information along with cytokine profile and transcriptome data are critical in the development of novel immune therapies. Real-time evaluation of single cells in response to antigen stimulation or others are valuable to evaluate the effectiveness of immune response. This is true for CD4 T cells as well. CAR-T cells that are proliferative, polyfunctional and which survive for longer periods are associated with better outcomes in oncology management. Similarly, macrophages may share transcriptome and epitope information, though they have different ability to secrete type of cytokines, growth factors, phagocytosis.

Conversely, characterizing cancer cells that are resistant to therapies (including cell therapy) are important to develop better strategies. This platform can also characterize single cells derived from clinical sample to determine prognosis and response to therapies.

We accomplish this by expanding the current features of Rhapsody cartridge and scanner as a platform to perform single cell functional assay and capture cell phenotype. Next, we identify the genotype, transcriptome and epitope information associated with cell function and phenotype with minimal changes to Rhapsody workflow by including Pheno-seq particles.

The methodology of the current invention finds use in a variety of different applications. Examples of such applications include, but are not limited to:
1. Characterizing cells based on their function is highly important to differentiate polyfunctional antigen specific CD4 and CD8 T cells and associated T cell receptor (TCR) sequence, transcriptome profile.
2. This is also important for other applications-such as characterizing CAR-T cells, T regulatory cells, Tumor associated macrophages, NK cells.
3. Other applications include characterizing differentiated cells from the population of stem cells—Cancer, regenerative medicine (based on their phenotype and function). This is very helpful to understand the development pathways in health and disease, mainly in cancer development, prognosis and outcomes.
4. Including functional and phenotype information along with CITE-seq will expand biomarker development applications.
5. Help characterizing B cells and identify sequence of antibodies involved in neutralizing viruses, Antibody dependent cell cytotoxicity (ADCC) and their effectiveness.
6. This methodology supports drug development activities where the effect of small molecules or therapeutics can be evaluated in multiple patient samples in a single experiment, and help correlate functional effectiveness of novel therapeutics, small molecules in multiple donors and identify genotype, transcriptome and epitope associated with degree of effectiveness. Each patient sample is identified by Sample Tag sequence, the cells are dispensed in Rhapsody wells to obtain single cells and treated with the small molecule of interest. 5000 to 10,000 single cells in a cartridge, can support evaluating effect in 50-100 cells of 100 patients. Additionally, samples from multiple Rhapsody cartridges can be combined to include in a single RNA-seq run.

Notwithstanding the appended claims, the disclosure is also defined by the following clauses:
1. A method of obtaining linked functional and sequence data for single cells of a cellular sample, the method comprising:
   contacting cells of the cellular sample with nucleic acid-barcoded, cell-binding beads to produce bead-bound cells;
   partitioning the bead-bound cells to produce partitioned bead-bound single cells each stably associated with a nucleic acid-barcoded cell-binding bead(s);
   functionally assaying the partitioned, bead-bound, single cells to obtain functional data for the partitioned, bead-bound, single cells;
   introducing unique combinations of distinct nucleic acid-barcoded identifying particles to partitions comprising functionally assayed bead-bound single cells to produce indexed partitions comprising:
      a functionally assayed bead-bound single cell; and
      a unique combination of nucleic acid-barcoded identifying particles;
   obtaining image data of the indexed partitions to identify the unique combination of nucleic acid-barcoded identifying particles therein;
   obtaining sequence data for barcode-comprising nucleic acids present in the partitions; and
   obtaining linked functional and sequence data for single cells of the cellular sample from the image data and sequence data.
2. The method according to Clause 1, wherein the nucleic acid-barcoded, cell-binding beads comprise:
   a bead;
   a specific binding member; and
   a cell-binding bead nucleic acid comprising a barcode.
3. The method according to Clause 2, wherein the cell-binding bead nucleic acid further comprises:
   a first domain complementary to a target binding region of a nucleic acid capture bead; and
   a second domain complementary to a sequence present in identifying particle nucleic acids of the nucleic acid-barcoded identifying particles.
4. The method according to any of Clauses 2 and 3, wherein the bead is magnetic.
5. The method according to any of the preceding clauses, wherein the specific binding member specifically binds to a cell surface marker.
6. The method according to Clause 5, wherein the specific binding member comprises an antibody or binding fragment thereof.
7. The method according to any of the preceding clauses, wherein the partitioning comprises distributing the bead-bound cells into partitions.
8. The method according to Clause 7, wherein the distributing comprises introducing the bead bound cells into a flow cell having microwells on a bottom surface thereof.
9. The method according to any of the preceding clauses, wherein functionally assaying the partitioned bead-bound single cells to obtain functional data for the partitioned bead-bound single cells comprises evaluating partitioned bead-bound single cells over time.
10. The method according to cany of the preceding clauses, wherein functionally assaying the partitioned bead-bound single cells to obtain functional data for the partitioned bead-bound single cells comprises evaluating partitioned bead-bound single cells in response to a stimulus.
11. The method according to Clause 10, wherein the stimulus is selected from the group consisting of chemical stimulus, mechanical stimulus, physical stimulus or combinations thereof.
12. The method according to any of the preceding clauses, wherein the unique combinations of distinct nucleic acid-barcoded identifying particles comprise a plurality of distinct nucleic acid-barcoded identifying particles that differ from each other by one or more of size, color and brightness.
13. The method according to any of the preceding clauses, wherein the number of distinct nucleic acid-barcoded identifying particles making up a combination in a partition ranges from 1 to 5.
14. The method according to any of the preceding clauses, wherein the distinct nucleic acid-barcoded identifying particles range in size from 3 to 20 μm.
15. The method according to any of the preceding clauses, wherein the distinct nucleic acid-barcoded identifying particles have colors selected from the group consisting of green, red, blue, grey, yellow and black.
16. The method according to any of the preceding clauses, wherein introducing unique combinations of distinct nucleic acid-barcoded identifying particles to partitions comprising functionally assayed bead-bound single cells comprises introducing a composition of distinct nucleic acid-barcoded identifying particles into a flow cell having microwells on a bottom surface thereof, wherein the microwells comprise functionally assayed bead-bound single cells.
17. The method according to Clause 16, wherein the composition of distinct nucleic acid-barcoded identifying particles comprises from 2 to 15 distinct nucleic acid-barcoded identifying particles.
18. The method according to Clauses 16 and 17, wherein the method further comprises introducing a second composition of distinct nucleic acid-barcoded identifying particles into the flow cell.
19. The method according to any of the preceding clauses, wherein the sequencing comprises providing a bead comprising a bead bound nucleic acid comprising cell label domain and a target binding region in the partitions comprising bead-bound single cells.
20. The method according to Clause 19, wherein the bead bound nucleic acid further comprises one or more of a molecular index domain and a universal primer binding domain.
21. The method according to any of the preceding clauses, wherein obtaining sequence data for the partitioned combinatorial barcoded single cells comprises employing a next generation sequencing protocol.
22. The method according to Clause 21, wherein the next generation sequencing protocol comprises producing a sequence ready library.
23. The method according to Clause 22, wherein producing the sequence ready library comprises a reverse transcription step and an amplification step.
24. The method according to Clause 23, wherein the amplification step comprises employing a first set of primers to amplify cell label barcode comprising nucleic acids and a second set of primers to amplify distinct nucleic acid-barcoded identifying particle nucleic acids.
25. The method according to any of the preceding clauses, wherein the sequencing data comprises multiomic data.
26. A composition of a plurality of distinct nucleic acid-barcoded identifying particles, wherein the plurality of distinct nucleic acid-barcoded identifying particles differ from each other by one or more of size, color and brightness.
27. The composition according to Clause 26, wherein the composition of distinct nucleic acid-barcoded identifying particles comprises from 2 to 15 distinct nucleic acid-barcoded identifying particles.
28. The composition according to Clauses 26 and 27, wherein the distinct nucleic acid-barcoded identifying particles range in size from 3 to 20 μm.
29. The composition according to Clauses 26 to 28, wherein the distinct nucleic acid-barcoded identifying particles have colors selected from the group consisting of green, red, blue, grey, yellow and black.
30. A kit for obtaining linked function and sequence data for single cells of a cellular sample, the kit comprising:
   a composition of a plurality of distinct nucleic acid-barcoded identifying particles, wherein the plurality of distinct nucleic acid-barcoded identifying particles differ from each other by one or more of size, color and brightness; and
   nucleic acid-barcoded cell-binding beads.
31. The kit according to Clause 30, wherein the composition of distinct nucleic acid-barcoded identifying particles comprises from 2 to 15 distinct nucleic acid-barcoded identifying particles.
32. The kit according to Clauses 30 and 31, wherein the distinct nucleic acid-barcoded identifying particles range in size from 3 to 20 μm.
33. The kit according to Clauses 30 to 32, wherein the distinct nucleic acid-barcoded identifying particles have colors selected from the group consisting of green, red, blue, grey, yellow and black.
34. The kit according to any of Clauses 30 to 33, wherein the nucleic acid-barcoded cell-binding beads comprise:
   a bead;
   a specific binding member; and
   a cell-binding bead nucleic acid comprising a barcode.
35. The kit according to Clause 34, wherein the cell-binding bead nucleic acid further comprises:
   a first domain complementary to a target binding region of a nucleic acid capture bead; and
   a second domain complementary to a sequence present in identifying particle nucleic acids of the nucleic acid-barcoded identifying particles.
36. The kit according to any of Clauses 34 and 35, wherein the bead is magnetic.
37. The kit according to any of the preceding clauses, wherein the specific binding member specifically binds to a cell surface marker.
38. The kit according to Clause 37, wherein the specific binding member comprises an antibody or binding fragment thereof.
39. The kit according to any of Clauses 30 to 38, wherein the kit further comprises beads comprising a bead bound nucleic acid comprising a cell label domain and a target binding region.
40. The kit according to any of Clauses 30 to 39, wherein the kit further comprises a flow cell having microwells on a bottom surface thereof.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of obtaining linked functional and sequence data for single cells of a cellular sample, the method comprising:
contacting cells of the cellular sample with nucleic acid-barcoded, cell-binding beads to produce bead-bound cells;
partitioning the bead-bound cells to produce partitioned bead-bound single cells each stably associated with a nucleic acid-barcoded cell-binding bead(s);
functionally assaying the partitioned, bead-bound, single cells to obtain functional data for the partitioned, bead-bound, single cells;
introducing unique combinations of distinct nucleic acid-barcoded identifying particles to partitions comprising functionally assayed bead-bound single cells to produce indexed partitions comprising:
a functionally assayed bead-bound single cell; and
a unique combination of nucleic acid-barcoded identifying particles;
obtaining image data of the indexed partitions to identify the unique combination of nucleic acid-barcoded identifying particles therein;
obtaining sequence data for barcode-comprising nucleic acids present in the partitions; and
obtaining linked functional and sequence data for single cells of the cellular sample from the image data and sequence data.

2. The method according to claim 1, wherein the nucleic acid-barcoded, cell-binding beads comprise:
a bead;
a specific binding member; and
a cell-binding bead nucleic acid comprising a barcode.

3. The method according to claim 2, wherein the cell-binding bead nucleic acid further comprises:
a first domain complementary to a target binding region of a nucleic acid capture bead; and
a second domain complementary to a sequence present in identifying particle nucleic acids of the nucleic acid-barcoded identifying particles.

4. The method according to claim 2, wherein the bead is magnetic.

5. The method according to claim 1, wherein the specific binding member specifically binds to a cell surface marker.

6. The method according to claim 5, wherein the specific binding member comprises an antibody or binding fragment thereof.

7. The method according to claim 1, wherein the partitioning comprises distributing the bead-bound cells into partitions.

8. The method according to claim 7, wherein the distributing comprises introducing the bead bound cells into a flow cell having microwells on a bottom surface thereof.

9. The method according to claim 1, wherein functionally assaying the partitioned bead-bound single cells to obtain functional data for the partitioned bead-bound single cells comprises evaluating partitioned bead-bound single cells over time.

10. The method according to claim 1, wherein functionally assaying the partitioned bead-bound single cells to obtain functional data for the partitioned bead-bound single cells comprises evaluating partitioned bead-bound single cells in response to a stimulus.

11. The method according to claim 1, wherein the unique combinations of distinct nucleic acid-barcoded identifying particles comprise a plurality of distinct nucleic acid-barcoded identifying particles that differ from each other by one or more of size, color and brightness.

12. The method according to claim 1, wherein introducing unique combinations of distinct nucleic acid-barcoded identifying particles to partitions comprising functionally assayed bead-bound single cells comprises introducing a composition of distinct nucleic acid-barcoded identifying particles into a flow cell having microwells on a bottom surface thereof, wherein the microwells comprise functionally assayed bead-bound single cells.

13. The method according to claim 1, wherein the sequencing comprises providing a bead comprising a bead bound nucleic acid comprising cell label domain and a target binding region in the partitions comprising bead-bound single cells.

14. The method according to claim 1, wherein obtaining sequence data for the partitioned combinatorial barcoded single cells comprises employing a next generation sequencing protocol.

15. The method according to claim 1, wherein the sequencing data comprises multiomic data.

16. A composition of a plurality of distinct nucleic acid-barcoded identifying particles, wherein the plurality of distinct nucleic acid-barcoded identifying particles differ from each other by one or more of size, color and brightness.

17. A kit for obtaining linked function and sequence data for single cells of a cellular sample, the kit comprising:
a composition of a plurality of distinct nucleic acid-barcoded identifying particles, wherein the plurality of distinct nucleic acid-barcoded identifying particles differ from each other by one or more of size, color and brightness; and
nucleic acid-barcoded cell-binding beads.
